(19) United States
(12) Patent Application Publication
(10) Pub. No.: US 2005/0180885 A1
(43) Pub. Date: Aug. 18, 2005

(54) Sheath flow cell cuvette, method of fabricating the same and flow cytometer including the same

(75) Inventors: Naohisa Tateishi, Hamamatsu-shi (JP); Yuji Masuda, Hamamatsu-shi (JP); Toshiaki Ushizu, Hamamatsu-shi (JP)

Correspondence Address: MORGAN LEWIS & BOCKIUS LLP, 1111 PENNSYLVANIA AVENUE NW, WASHINGTON, DC, 20004, US

(73) Assignee: HAMAMATSU PHOTONICS K.K.

(21) Appl. No.: 11/030,319

(22) Filed: Jan. 7, 2005

(30) Foreign Application Priority Data
Jan. 9, 2004 (JP) JP2004-004300

(51) Int. Cl. G01N 33/00

(52) U.S. Cl. 422/68.1; 435/287.1

(57) ABSTRACT

The present invention relates to a sheath flow cell cuvette and the like provided with a structure to effectively prevent relative positional fluctuation between component members. The said sheath flow cell cuvette comprises a chamber portion comprised of a resin and an orifice portion comprised of a glass material. One end of the orifice portion is buried in the chamber portion, and at this buried part, a latching structure to prevent the orifice portion from shifting with respect to the chamber portion is provided. A cell suspension fluid of a measuring object is injected at high pressure from the chamber portion toward the orifice portion while being surrounded by a sheath fluid. At this time, although an extruding pressure along a flowing direction of the cell suspension fluid is exerted on the orifice portion, since a relative positional fluctuation between the chamber portion and orifice portion is avoided by an action of the latching structure covered with the resin of a part of the chamber portion, a laminar flow condition between the cell suspension fluid and sheath fluid is stably maintained.

### BACKGROUND OF THE INVENTION

1. Field of the Invention

The present invention relates to a sheath flow cell cuvette applicable to a flow cytometer, a method of fabricating the sheath flow cell cuvette, and flow cytometer including the sheath flow cell cuvette.

2. Related Background of the Invention

For example, in the medical field and the like, when examining and analyzing cells in blood, urine and the like, a flow cytometer for electrical and optical measurement is used. By this flow cytometer, by flowing a sheath fluid around a cell suspension fluid of blood, urine or the like or a cell suspension fluid wherein these have been dyed with an appropriate stain, the cell suspension fluid is narrowed down in a sheath flow cell cuvette. The sheath flow cell cuvette comprises, for example, a chamber portion made of a resin (rectifying portion) and an orifice portion (detecting portion) made of silica glass whose one end has been joined by an adhesive with a chamber. In said sheath flow cell cuvette, a cell suspension fluid narrowed down by a sheath fluid is made to flow from the chamber portion to the orifice portion at high speed, and an electrical and optical measurement is carried out for this cell suspension fluid flowing inside the orifice portion. As sheath flow cell cuvettes applicable to such a flow cytometer, sheath flow cell cuvettes disclosed in, for example, Japanese Patent No. 2874746 and Japanese Patent Application Laid-Open No. 2002-31595 have been known. In addition, for the sheath flow cell cuvettes, in order to eliminate discrepancies in measurement, a laminar flow condition of the cell suspension fluid and sheath fluid (a condition of the cell suspension fluid flowing while being surrounded by the sheath fluid) is required, thus it is necessary that the inner circumferential surfaces between the chamber portion and orifice portion are smoothly joined.

### SUMMARY OF THE INVENTION

As a result of an investigation on the conventional sheath flow cell cuvette as described above, the inventor has discovered the following problems.

Namely, for a sheath flow cell cuvette, in order to measure a large number of cell suspension fluids in a short time and obtain accurate data, it is necessary to inject the cell suspension fluids at high pressure. In this case, there is a possibility that the orifice portion is displaced with respect to the chamber portion by a high-pressure injected cell suspension fluid in a flowing direction of the cell suspension fluid. Once the orifice portion is displaced with respect to the chamber portion, a laminar flow condition between the cell suspension fluid and sheath fluid is not maintained, and accurate data is no longer obtained.

The present invention has been made to solve the problem as described above, and it is an object of the invention to provide a sheath flow cell cuvette with a structure to effectively prevent a shift of an orifice portion with respect to a chamber portion in a cell suspension fluid flowing direction, a method of fabricating the sheath flow cell cuvette, and a flow cytometer including the sheath flow cell cuvette.

A sheath flow cell cuvette according to the present invention is applicable to a flow cytometer to carry out an electrical and optical measurement for cells in blood, urine or the like, and functions so that, by making a sheath fluid flow around a cell suspension fluid of a measuring object, the cell suspension fluid is narrowed down.

In order to realize such a function as described above, a sheath flow cell cuvette according to the present invention comprises a chamber portion comprised of a resin as a rectifying portion and an orifice portion comprised of a glass material as a detecting portion. One end of the orifice portion is buried in the chamber portion, and at this buried part, a latching structure to prevent the orifice portion from shifting in a flowing direction of the cell suspension fluid with respect to the chamber portion is provided.

In accordance with the sheath flow cell cuvette having such a structure as described above, since a relative positional fluctuation (a shift along a flowing direction of the cell suspension fluid) between the orifice portion and chamber portion is avoided by a latching structure provided at the buried part of the orifice portion, the orifice portion is securely fitted to the chamber portion, thus a laminar flow condition between the cell suspension fluid and sheath fluid is stably maintained.

In the sheath flow cell cuvette according to the present invention, the above-described latching structure can be constituted by at least one of a concave portion and a convex portion. Thereby, the orifice portion is securely fixed to the chamber portion. In addition, the latching structure may be constructed so as to have a larger outside diameter than an outside diameter in a region other than the buried part of the orifice portion. In this case as well, the orifice portion is securely fixed to the chamber portion.

Also, in the sheath flow cell cuvette having such a structure as described above, since the chamber portion to cover the latching structure in the orifice portion is made of a resin, various fabricating methods (a sheath flow cell cuvette fabricating method according to the present invention) can be applied thereto.

Namely, in a sheath flow cell cuvette fabricating method according to the present invention, a mold having an inner surface corresponding to contours of the chamber portion is prepared. On the prepared mold, a core whose front end has been conically processed having a shape corresponding to the first through-hole of the chamber portion, and while making one end of the orifice portion to be a buried part on which a latching structure has been provided proceed to the inner surface of the mold, the one end of the orifice portion is made in contact with the conical front end of the core. In such a condition, a resin is filled into the mold. And, the mold is removed after the filled resin solidifies, whereby a sheath flow cell cuvette having a structure as described above is obtained.

By such a sheath flow cell cuvette fabricating method, since a resin is filled into the mold while the front end (conical shape) of the core and one end of the orifice portion are in contact, the chamber portion and orifice portion are integrally molded. Thereby, a sheath flow cell cuvette having a structure as described above is obtained, and an inner circumferential surface of the chamber portion and orifice portion is made as a smooth and continuous inner circumferential surface.

On the other hand, said sheath flow cell cuvette is also obtained by hot-forming a resin. Namely, an inner mold having a shape corresponding to the first through-hole of the chamber portion and whose front end has been conically processed is prepared. On the other hand, a region (the above-described latching structure has been formed in advance) to be a buried part of the orifice portion is covered with a heat shrinkable tube which shrinks by heating. And, while the inner mold is made to proceed inside the heat shrinkable tube so that a front-end part makes contact with one end of the orifice portion on which the latching structure has been provided, said heat shrinkable tube is heated. This heated part becomes a chamber portion. Namely, the inner mold is removed from the heated heat shrinkable tube, whereby a sheath flow cell cuvette having such a structure as described above is obtained.

By such a sheath flow cell cuvette fabricating method, the heat shrinkable tube covering the latching structure is heated while the conical front end of the inner mold and one end of the orifice portion are in contact. With this heated heat shrinkable tube being as a chamber portion, the chamber portion and orifice portion are integrally molded. Therefore, a sheath flow cell cuvette having such a structure as described above is easily obtained, and an inner circumferential surface of the chamber portion and orifice portion becomes a smooth and continuous inner circumferential surface.

A flow cytometer according to the present invention comprises a sheath flow cell cuvette having such a structure as described above, an introduction portion, an injection valve, and a measurement system. The introduction portion has a third through-hole communicated with the first through-hole of the chamber portion, and functions so as to introduce a sheath fluid into the first through-hole via the third through-hole. Here, this introduction portion having a third through-hole and the chamber portion having a first through-hole may be constructed as separate members, and may be integrally constructed. The injection valve is arranged so that a front end thereof is positioned in the first through-hole of the chamber portion, and functions so as to introduce the cell suspension fluid into the first through-hole via an opening provided in said front end. In addition, the measurement system electrically or optically obtains predetermined physical property data from the cell suspension fluid flowing inside the second through-hole of the orifice portion.

When the cell suspension fluid is optically measured, it is preferable that the measurement system comprises a light source to output light having a predetermined wavelength and a detecting portion for receiving light from the light source passed through the orifice portion. In particular, when an optical measurement is carried out as such, in order to avoid an irregular reflection on a light incidence plane in the orifice portion, it is preferable that the orifice portion has a shape of a square cylinder form extending along the second through-hole. As a result of a vertical irradiation of light from the light source onto a flat surface of the orifice portion, an irregular reflection of the light is efficiently avoided.

Here, respective embodiments according to the present invention will be more fully understood by the following detailed description and attached drawings. It should be regarded that these embodiments are merely illustrative and do not limit the present invention.

In addition, a further scope of applicability of the present invention will become apparent from the detailed description given hereinafter; however, it is to be understood that the detailed description and specific examples, while indicating preferred embodiments of the present invention, are given by way of mere illustration, and it is obvious that various changes and modifications within the spirit and scope of the invention are self-evident to those skilled in the art from the detailed description.

### BRIEF DESCRIPTION OF THE DRAWINGS

FIG. 1 is a view showing a sectional configuration of a flow cytometer (flow cytometer according to the present invention) to which a first embodiment of a sheath flow cell cuvette according to the present invention has been applied;

FIG. 2 is a partially broken view showing a latching structure in the sheath flow cell cuvette according to the first embodiment shown in FIG. 1;

FIG. 3 is a view for explaining a method of fabricating the sheath flow cell cuvette according to the first embodiment shown in FIG. 1, wherein a core arranged on a lower die is shown;

FIG. 4 is a view for explaining a step subsequent to the step shown in FIG. 3, wherein a lower die and an upper die before being filled with resin are shown;

FIG. 5 is a partial broken view showing a latching structure in a second embodiment of a sheath flow cell cuvette according to the present invention;

FIG. 6 is a partial broken view showing a latching structure in a third embodiment of a sheath flow cell cuvette according to the present invention; and

FIG. 7 is a view for explaining a method of fabricating a fourth embodiment of a sheath flow cell cuvette according to the present invention.

### DESCRIPTION OF THE PREFERRED EMBODIMENTS

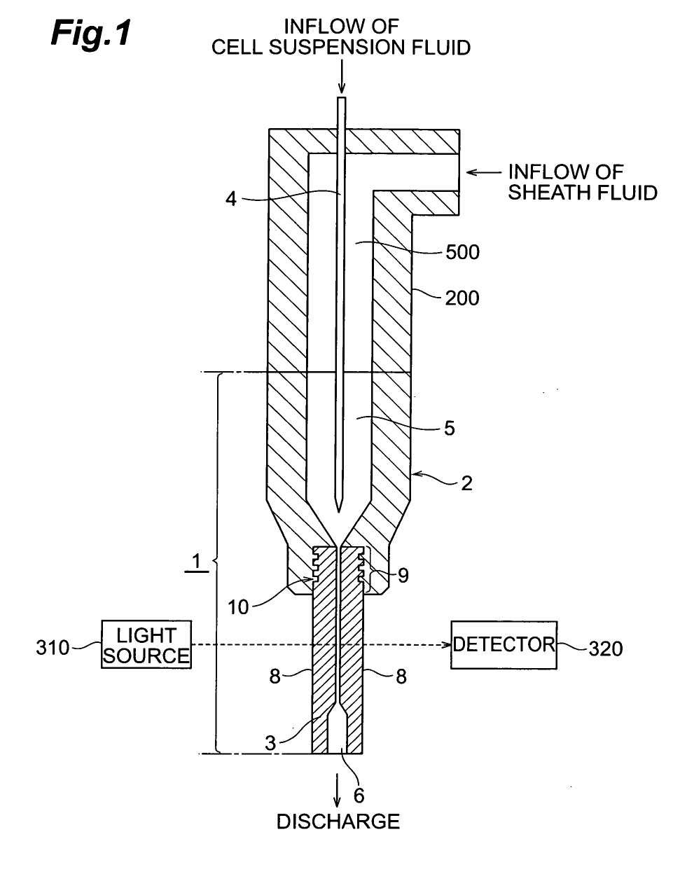

Hereinafter, respective embodiments of a sheath flow cell cuvette, a flow cytometer, and a fabricating method by the present invention will be described in detail by use of FIG. 1 to FIG. 7. Here, in the description of the drawings, identical symbols are used for identical elements, whereby overlapping description will be omitted.

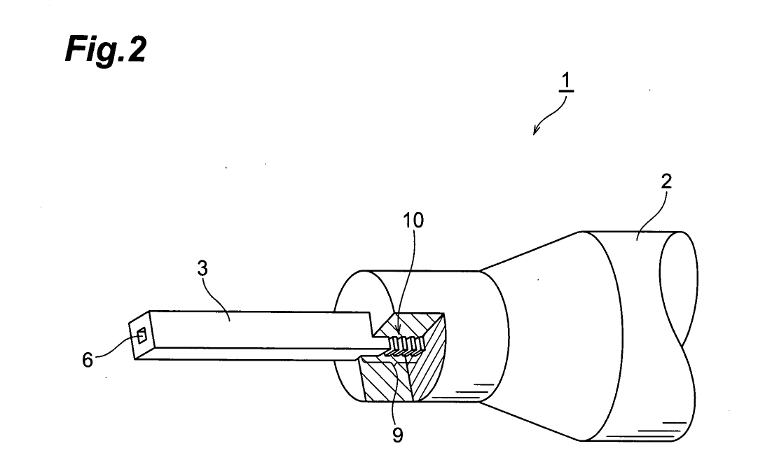

FIG. 1 is a view showing a sectional configuration of a flow cytometer to which a first embodiment of a sheath flow cell cuvette according to the present invention has been applied. In addition, FIG. 2 is a partially broken view showing a latching structure in the sheath flow cell cuvette according to the first embodiment shown in FIG. 1.

The flow cytometer shown in FIG. 1 is an apparatus, such as a blood analyzer, to measure a cell suspension fluid electrically and optically. Such a flow cytometer comprises a sheath flow cell cuvette 1, an introduction portion 200 for introducing a sheath fluid, an injection valve 4 for introducing a cell suspension fluid, and a measurement system.

The sheath flow cell cuvette applied to such a flow cytometer comprises a circular cylindrical chamber portion 2 and a cylindrical orifice portion 3 of a square cylinder form whose transverse section is regular square or rectangular. In addition, to the chamber portion 2, the introduction portion 2 having a through-hole 500 for introducing a sheath fluid is fixed so that mutual through-holes 5 and 500 are communicated. The injection valve 4 is arranged so that its front end is positioned in the through-hole 5 of the chamber portion 2, and a cell suspension fluid injected at high pressure from this front end of the injection valve 4 is narrowed by the sheath fluid. Here, the measurement system in the flow cytometer shown in FIG. 1 is, in order to enable an optical measurement, composed of a light source 310 for outputting light having a predetermined wavelength and a detector 320 for receiving light from the light source 310.

The chamber portion 2 is made of a resin with water resistance and chemical resistance, for example, polyester or the like, whose outside diameter on a cell suspension fluid inflow side (introduction portion side) is made as a large diameter, whose outside diameter on an outflow side (orifice portion side) is made as a small diameter, and is provided inside with a through-hole 5 which is circular in the transverse section along the longitudinal section. Here, in the flow cytometer shown in FIG. 1, although the introduction portion 200 and chamber portion 2 are shown as separate members, these may be integrally constructed.

The orifice portion 3 is made of, for example, a synthetic silica glass or the like, and is provided inside with a through-hole 6 along the longitudinal direction. This through-hole 6 is arranged coaxially with the through-hole 5 of the chamber portion 2, whose cell suspension fluid inflow side (chamber portion side) is continuous from the tapered through-hole 5 of the chamber portion 2, whose outflow side (discharge port side) is made as a large diameter, and whose inflow-side end to the large diameter portion of the outflow side is a continuous square hole with an identical sectional area. And, by this through-hole 6 and the through-hole 5 of the chamber portion 2, a smooth and continuous path (a path through which a cell suspension fluid flows) is constructed.

For this orifice portion 3, a laser light to measure the cell suspension fluid flowing inside the through-hole 6 is irradiated from the laser source 310, and opposed wall surfaces 8 and 8 are parallel so that the detector portion 320 can efficiently receive a forward-scattered light, which is a scattered light and a refracted light that occur on the cell surface and which scatters forward with respect to the axis of the laser light, and a lateral-scattered light, which is a scattered light that occurs in the nucleus in a cell and which scatters at an approximately right angle with respect to the axis of the laser light. These wall surfaces 8 are, in order to prevent energy loss of a transmitting light, flat surfaces.

As shown in FIG. 1 and FIG. 2, an end portion (equivalent to a buried part 9) of the orifice portion 3 positioned on the chamber portion 2 side is buried inside the chamber portion 2.

For this buried part 9, a latching structure 10 is provided on its outer circumference. The latching structure 10 is a plurality of latching grooves juxtaposed along a flow direction of the cell suspension fluid, and into these latching grooves, the resin of a part of the chamber portion 2 intrudes.

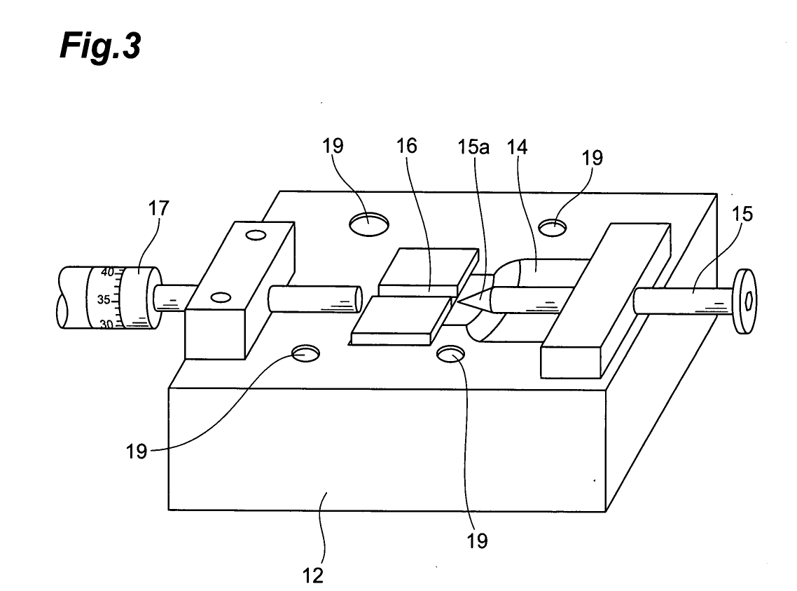

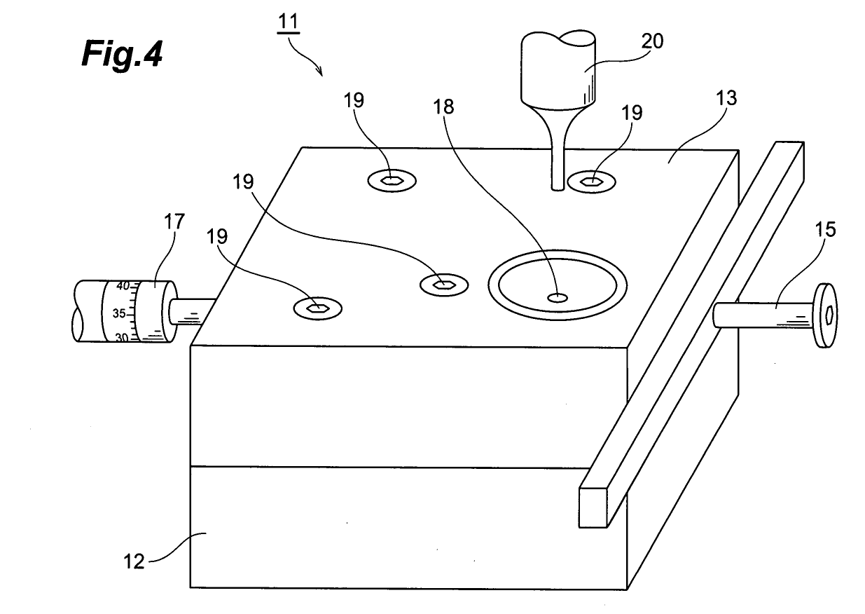

A method of fabricating the sheath flow cell cuvette 1 constructed as such will be described in the following. First, as shown in FIG. 4, a metal mold 11 is prepared as forming dies. Here, only a lower die 12 is shown in FIG. 3. The metal mold 11 comprises an upper die 13 and the lower die 12. These lower and upper dies 12 and 13 have an inner surface 14 corresponding to contours of the chamber portion 2.

On the lower die 12, a core 15 to form the through-hole 5 of the chamber portion 2, an orifice placing portion 16 on which the orifice portion 3 is placed, and a micrometer 17 are linearly disposed. The core 15 is a columnar body whose front end 15a has been conically processed, and this is disposed so as to be removed by pulling from the lower die 12.

The upper die 13 comprises, as shown in FIG. 4, a filling hole 18 communicated with the inner surface 14 to externally fill a resin. In these lower and upper dies 12 and 13, as shown in FIG. 3 and FIG. 4, screw holes 19 to pressure-fit and fix the metal dies 12 and 13 to each other are provided at predetermined positions.

And, in the metal mold 11 having such a shape, the orifice portion 3 is placed on the orifice placing portion 16, and the micrometer 17 makes the latching structure 10 of the orifice portion 3 proceed to the inner surface 14 of the metal mold 11. Namely, the micrometer 7 makes the orifice portion 3 shift until the front end of the conical body 15a of the core 15 is brought in contact with the through-hole 6 of the orifice portion 3. Next, as shown in FIG. 4, the upper die 13 is placed over the lower die 12, and the metal dies 12 and 13 are pressure-fitted and fixed to each other by screws.

Next, a heated resin (heated inside an unillustrated tank) is filled via a filling nozzle 20 and the filling hole 18 into a space formed by the inner surface 14 of the metal mold 11, and the filled resin solidifies as a result of heat radiation by the metal mold 11. After resin filling is completed, the above-mentioned sheath flow cell cuvette 1 is obtained by removing the solidified resin from the metal mold 11. Here, in order to ease mold releasing, the core 15 has a slightly tapered shape at its outer circumference.

In such a sheath flow cell cuvette 1, by the latching structure on the outer circumference of the buried part 9 of the orifice portion 3 buried in the chamber portion 2, the orifice portion 3 is securely fixed to the chamber portion 2. Accordingly, a shift of the orifice portion 3 in the flow direction of the cell suspension fluid with respect to the chamber portion 2 is efficiently prevented, thus a laminar flow condition of the cell suspension fluid and sheath fluid is stably maintained. As a result, it becomes possible to provide a high-quality sheath flow cell cuvette 1.

Additionally, in accordance with the fabricating method for a sheath flow cell cuvette 1 as described above, while the front end 15a (conical body) of the core 15 and one end (equivalent to the buried part 9) of the orifice portion 3 are in contact, a resin is filled in the inner surface 14 of the metal mold 11. Thereby, the chamber portion 2 and orifice portion 3 are integrally molded, the above-described sheath flow cell cuvette 1 is easily obtained, and moreover, an inner circumferential surface defined by the through-holes 5 and 6 of the chamber portion 2 and orifice portion 3 is made smooth and continuous. As a result, it is made possible to provide a fabricating method for a high-quality sheath flow cell cuvette 1. Incidentally, in this fabricating method for a sheath flow cell cuvette 1 according to the first embodiment, a favorable sheath flow cell cuvette 1 is obtained with a resin filling time of 20 minutes, a filling pressure of 30 kg/cm², a tank temperature of 220° C., and a filling nozzle temperature of 230°.

As in the prior art, when the chamber portion and orifice portion are joined by an adhesive, since the chamber portion and orifice portion have been separately manufactured, respectively, these cannot correspond to variations in shape, and a gap occurring at a joint portion even if these are fitted together by use of jigs or the like. However, according to the present first embodiment, individual subtle changes in shape are absorbed by integral molding, and a smooth and continuous inner circumferential surface is defined by the through-holes 5 and 6 of the respective members 2 and 3. As a result, yield is improved, which makes it possible to reduce the fabricating cost of a sheath flow cell cuvette 1.

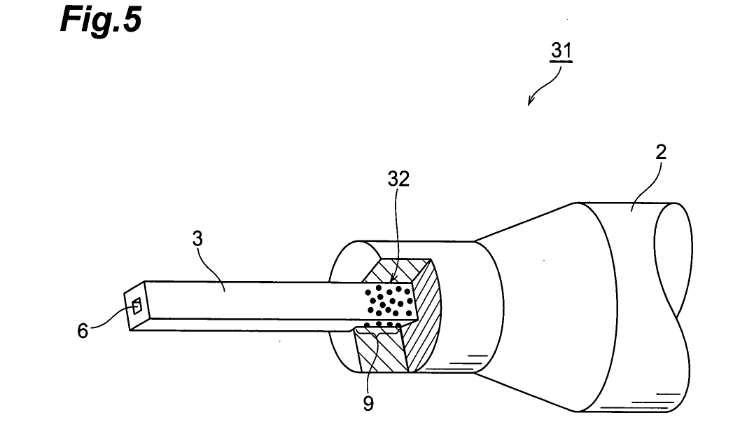

FIG. 5 is a partial broken view showing a latching structure in a second embodiment of a sheath flow cell cuvette according to the present invention. This sheath flow cell cuvette 31 according to the second embodiment is different from the sheath flow cell cuvette 1 according to the first embodiment in that, in place of the latching structure 10 composed of latching grooves, a latching structure 32 is composed of a plurality of point-like projection (salients). Similar to the first embodiment by such a latching structure 32, as well, the orifice portion 3 is securely fixed to the chamber portion 2.

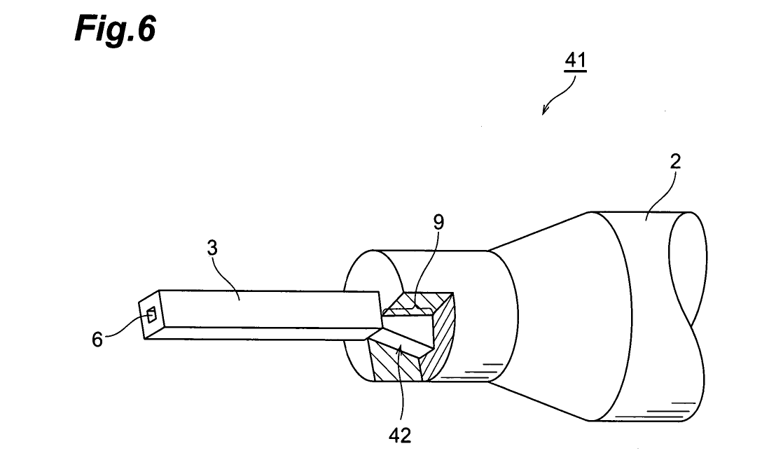

FIG. 6 is a partial broken view showing a latching structure in a third embodiment of a sheath flow cell cuvette according to the present invention. This sheath flow cell cuvette 41 according to the third embodiment is different from the sheath flow cell cuvette 1 according to the first embodiment in that, in place of the latching structure 10 composed of latching grooves, a latching structure 42 is formed of a large diameter portion. This large diameter portion has a greater diameter than an outside diameter of a region other than the buried part 9 of the orifice portion 3, and is, in this third embodiment, in a truncated quadrangular pyramid form which has a small diameter at the burying border. Similar to the first embodiment by such a latching structure 42, as well, the orifice portion 3 is securely fixed to the chamber portion 2.

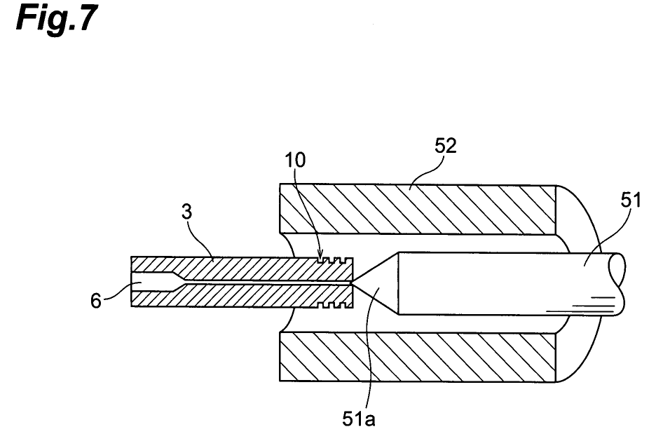

FIG. 7 is a view for explaining a method of fabricating a fourth embodiment of a sheath flow cell cuvette according to the present invention. Although this sheath flow cell cuvette according to the fourth embodiment is the same in shape as the sheath flow cell cuvette 1 according to the first embodiment shown in FIG. 1 and FIG. 2, this is different in its fabricating method. Concretely, first, an inner mold 51 of a columnar body whose front end 51a has been conically processed is prepared, and the orifice portion 3 is fixed while the through-hole 6 of the orifice portion 3 is in contact with the front end 51a of this inner mold 51. Next, the latching structure 10 and inner mold 51 are covered with a heat shrinkable tube 52, and this heat shrinkable tube 52 is heated by a hot-air heater. Then, the heat shrinkable tube 52 shrinks, intrudes into latching grooves composing the latching structure 10, and makes close contact with the inner mold 51. Then, after shrinkage of the heat shrinkable tube 52 is completed, the above-described sheath flow cell cuvette 1 is obtained by removing the inner mold 51 from the heat shrinkable tube.

Similar to the first embodiment by such a fabricating method, as well, a sheath flow cell cuvette to provide the above-described effects can be easily obtained, and an inner circumferential surface defined by the through-holes 5 and 6 of the chamber portion 2 and orifice portion 3 is made smooth and continuous.

As in the above, the present invention has been concretely described based on embodiments thereof, however, the present invention is not limited to the embodiments as described above, the latching structures 10, 32, and 42 may be provided as helicoidal latching grooves, and may also be various types of convex portions, concave portions, uneven portions, linear forms, and curved forms. In short, it is sufficient that these are structures whose engagement with a resin is excellent so that relative positional fluctuation of the orifice portion 3 with respect to the chamber portion 2 can be effectively avoided.

In addition, in the embodiments as described above, although polyester has been used as the material of the chamber portion 2, it may be polycarbonate, Teflon (trade name) or the like, for example, and in short, it is sufficient that it is a resin with water resistance and chemical resistance.

Furthermore, in the embodiments as described above, although synthetic silica glass has been used as being particularly preferable as the material of the orifice portion 3, it may be another type of silica glass, for example, and in short, a glass material is sufficient.

In the embodiments as described above, although the through-hole 5 of the chamber portion 2 has been circular in the transverse section, it may be elliptic or the like, for example.

The through-hole 6 of the orifice portion 3 may be, without providing a large diameter portion on the cell suspension fluid outflow side, a square hole continuing from the cell suspension fluid inflow side to the outflow side with an identical sectional area, or may be a conical shape whose sectional diameter is reduced toward the outflow side.

As in the above, by a sheath flow cell cuvette according to the present invention, since a laminar flow condition of the cell suspension fluid and sheath fluid are maintained as a result of a relative positional fluctuation of the orifice portion 3 with respect to the chamber portion being effectively avoided, a high-quality sheath flow cell cuvette can be obtained.

In addition, by a fabricating method thereof, a sheath flow cell cuvette to provide the above-described effects is easily obtained, and since an inner circumferential surface defined by the through-holes of the chamber portion and orifice portion is made smooth and continuous, a sheath flow cell cuvette wherein a laminar flow condition of the cell suspension fluid and sheath fluid are stably maintained can be easily obtained, thereby it becomes possible to provide a fabricating method for a high-quality sheath flow cell cuvette.

From the above description of the present invention, it is obvious that the present invention can be variously modified. Such modifications cannot be regarded as departing from the spirit and scope of the invention, and all improvements self-evident to those skilled in the art are to be included in the following claims.

1. A sheath flow cell cuvette, comprising:
a chamber portion having a first through-hole into which a cell suspension fluid is introduced along with a sheath fluid and comprised of a resin; and
an orifice portion having a second through-hole communicated with the first through-hole of said chamber portion and comprised of a glass material, said orifice portion having one end buried inside said chamber portion and being provided, at the buried part of said orifice portion, with a latching structure to prevent said orifice portion from shifting in a flowing direction of the cell suspension fluid with respect to said chamber portion.

2. A sheath flow cell cuvette according to claim 1, wherein said orifice portion has a shape of a square cylinder form extending along the second through-hole.

3. A sheath flow cell cuvette according to claim 1, wherein said latching structure provided at the buried part of said orifice portion is constituted by at least one of a concave portion and a convex portion.

4. A sheath flow cell cuvette according to claim 1, wherein said latching structure provided at the buried part of said orifice portion has a larger outside diameter than an outside diameter of a part not buried in said orifice portion.

5. A method of fabricating a sheath flow cell cuvette according to claim 1, comprising the steps of:
preparing a mold having an inner surface corresponding to contours of said chamber portion;
arranging, on said prepared mold, a core whose front end has been conically processed having a shape corresponding to said first through-hole of said chamber portion;
bring the one end of said orifice portion into contact with said conical front end of said core, while making one end of said orifice portion to be a buried part on which a latching structure has been provided proceed to the inner surface of said mold;
filling a resin into said mold; and
obtaining said sheath flow cell cuvette, by removing the mold after the filled resin solidifies.

6. A method of fabricating a sheath flow cell cuvette according to claim 1, comprising the steps of:
preparing an inner mold having a shape corresponding to said first through-hole of said chamber portion and whose front end has been conically processed;
covering a latching structure provided in a region to be a buried part of said orifice portion with a heat shrinkable tube which shrinks by heating;
making said inner mold proceed inside said heat shrinkable tube so that a front-end part makes contact with one end of said orifice portion on which said latching structure has been provided;
forming said chamber portion by heating said heat shrinkable tube; and
obtaining a sheath flow cell cuvette, by removing said inner mold from said heated heat shrinkable tube.

7. A flow cytometer, comprising:
a sheath flow cell cuvette according to claim 1;
an introduction portion having a third through-hole communicated with said first through-hole of said chamber portion, for introducing a sheath fluid into said first through-hole via said third through-hole;
an injection valve arranged so that a front end thereof is positioned in said first through-hole of said chamber portion, for introducing the cell suspension fluid into said first through-hole via an opening provided in said front end; and
a measurement system for obtaining predetermined physical property data from the cell suspension fluid flowing inside said second through-hole of said orifice portion.
